(19) United States
(12) Patent Application Publication
(10) Pub. No.: US 2026/0199271 A1
(43) Pub. Date: Jul. 16, 2026

(54) ACIDIC CANNABINOIDS AND USES THEREOF FOR ENHANCING FEMALE SEXUAL FUNCTION OR TREATING FEMALE SEXUAL DISORDERS

(71) Applicant: Vella Bioscience, Inc., Worcester, MA (US)

(72) Inventors: Michael Frid, Medford, MA (US); Harin Padma-Nathan, Los Angeles, CA (US); Nial C. DeMena, Worcester, MA (US)

(21) Appl. No.: 19/439,620

(22) Filed: Jan. 5, 2026

Related U.S. Application Data
(63) Continuation of application No. 18/120,725, filed on Mar. 13, 2023, now abandoned.
(60) Provisional application No. 63/321,714, filed on Mar. 20, 2022.

(51) Int. Cl. A61K 31/19; A61K 31/00; A61K 31/417; A61K 36/185; A61K 45/06; A61P 15/02

(52) U.S. Cl. CPC A61K 31/19; A61K 31/417; A61K 31/658; A61K 36/3482; A61K 45/06; A61P 15/02

(57) ABSTRACT

The present disclosure provides peripherally acting cannabidiolic acid (CBDA)-containing compositions and methods of using thereof for treating or enhancing female sexual function. In some embodiments, the compositions are provided in the form of a topical dosage form, which is applied to female genitalia shortly prior to sexual activity.

### RELATED APPLICATION(S)

This application is a continuation of U.S. application Ser. No. 18/120,725, filed Mar. 13, 2023, which claims the benefit of U.S. Provisional Application No. 63/321,714, filed on Mar. 20, 2022.

The entire teachings of the above application(s) are incorporated herein by reference.

### FIELD OF THE INVENTION

The invention relates to compositions containing acidic cannabinoid(s) and uses thereof in enhancing female sexual function or treating or ameliorating certain female sexual disorders.

### BACKGROUND

Female sexual dissatisfaction, defined herein as unsatisfactory sexual function in healthy women, and dysfunction, defined herein as a diagnosable condition within the definition of Female Sexual Disorders in The Diagnostic and Statistical Manual of Mental Disorders-5 (DSM-5), are highly prevalent and their incidence and prevalence are likely underestimated. In a recent study of young women, presumably the healthiest segment of the female population, prevalence of sexually related personal distress was 50.2%, wherein sexually related personal distress without dysfunction affected 29.6%, and 20.6% had at least one female sexual dysfunction (Zheng, J., Skiba, M. A., Bell, R. J., Islam, R. M., and Davis, S. R. (2020) The prevalence of sexual dysfunctions and sexually related distress in young women: a cross-sectional survey, Fertility and Sterility 113, 426-434). Personal distress is defined herein as feelings of frustration, grief, incompetence, loss, sadness, sorrow, or worry (Parish, S. J. et al. (2016) Toward a More Evidence-Based Nosology and Nomenclature for Female Sexual Dysfunctions-Part II, Journal of Sexual Medicine 13, 1888-1906). Age, physical fitness, stress, partner non-performance, and a variety of other causes may contribute to female sexual dissatisfaction, among which negative body image has been found to correlate strongly with sexual satisfaction, including aspects of arousal and orgasm (Woertman, L., and van den Brink, F. (2012) Body image and female sexual functioning and behavior: a review, Journal of Sex Research 49, 184-211). Female sexual dysfunction is common in the United States and worldwide. Over 40% of women in Western countries reported experiencing sexual problems (Laumann, E. O., Paik, A., and Rosen, R. C. (1999) Sexual dysfunction in the United States: prevalence and predictors, Journal of American Medical Association 281, 537-544; Shifren, J. L., Monz, B. U., Russo, P. A., Segreti, A., and Johannes, C. B. (2008) Sexual problems and distress in United States women: prevalence and correlates, Obstetrics & Gynecology 112, 970-978; Hendrickx, L., Gijs, L., and Enzlin, P. (2014) Prevalence rates of sexual difficulties and associated distress in heterosexual men and women: results from an Internet survey in Flanders, Journal of Sex Research 51, 1-12; Hendrickx, L., Gijs, L., and Enzlin, P. (2015) Age-related prevalence rates of sexual difficulties, sexual dysfunctions, and sexual distress in heterosexual women: results from an online survey in Flanders, Journal of Sexual Medicine 12, 424-435; Wolpe, R. E., Zomkowski, K., Silva, F. P., Queiroz, A. P. A., and Sperandio, F. F. (2017) Prevalence of female sexual dysfunction in Brazil: A systematic review, European Journal of Obstetrics & Gynecology and Reproductive Biology 211, 26-32; McCabe, M. P., Sharlip, I. D., Lewis, R., Atalla, E., Balon, R., Fisher, A. D., Laumann, E., Lee, S. W., and Segraves, R. T. (2016) Incidence and Prevalence of Sexual Dysfunction in Women and Men: A Consensus Statement from the Fourth International Consultation on Sexual Medicine 2015, Journal of Sexual Medicine 13, 144-152; (2019) Female Sexual Dysfunction: ACOG Practice Bulletin Clinical Management Guidelines for Obstetrician-Gynecologists, Number 213, Obstetrics & Gynecology 134, e1-e18).

There is ample evidence for the negative effects the ongoing SARS-Cov-2 (COVID) pandemic has had on the mental health of American adults (Holman, E. A., Thompson, R. R., Garfin, D. R., and Silver, R. C. (2020) The unfolding COVID-19 pandemic: A probability-based, nationally representative study of mental health in the United States, Science Advances 6, eabd5390; Ettman, C. K., Abdalla, S. M., Cohen, G. H., Sampson, L., Vivier, P. M., and Galea, S. (2020) Prevalence of Depression Symptoms in US Adults Before and During the COVID-19 Pandemic, JAMA Network Open 3, e2019686). The same pattern has been observed around the globe (COVID—Mental Disorders Collaborators. (2021) Global prevalence and burden of depressive and anxiety disorders in 204 countries and territories in 2020 due to the COVID-19 pandemic, Lancet. Published Online Oct. 8, 2021). Notably, the negative mental health consequences of the COVID pandemic have impacted the young females most strongly (Minhas, M., Belisario, K., González-Roz, A., Halladay, J., Murphy, J. G., and MacKillop, J. (2021) COVID-19 impacts on drinking and mental health in emerging adults: Longitudinal changes and moderation by economic disruption and sex, Alcoholism: Clinical and Experimental Research 45, 1448; Stephenson, J. (2021) CDC Study Finds Worsening Anxiety and Depression, Especially in Young Adults, During COVID-19 Pandemic, JAMA Health Forum 2, e210724; Vahratian, A., Blumberg, S. J., Terlizzi, E. P., Schiller, J. S. Symptoms of Anxiety or Depressive Disorder and Use of Mental Health Care Among Adults During the COVID-19 Pandemic—United States, August 2020-February 2021. MMWR Morbidity and Mortality Weekly Report 2021, 70:490; Jia, H., Guerin, R. J., Barile, J. P., et al. National and State Trends in Anxiety and Depression Severity Scores Among Adults During the COVID-19 Pandemic—United States, 2020-2021. MMWR Morbidity and Mortality Weekly Report 2021, 70:1427). The increase in incidence and prevalence of depressive spectrum disorders likely has translated into a corresponding increase in the incidence and prevalence of female sexual dysfunction because the positive association between depression and female sexual dysfunction has been established (Basson, R., and Gilks, T. (2018) Women's sexual dysfunction associated with psychiatric disorders and their treatment, Women's Health 14, 1-16), and a bidirectional, reinforcing relationship was shown to exist between depression and sexual dysfunction (Atlantis, E., and Sullivan, T. (2012) Bidirectional Association between Depression and Sexual Dysfunction: A Systematic Review and Meta-Analysis, Journal of Sexual Medicine 9, 1497-1507). Furthermore, the pharmacological treatments of depression also correlate positively with female sexual dysfunction (Clayton, A. H., et al. (2014). Antidepressants and sexual dysfunction: mechanisms and clinical implications. Postgraduate Medicine 126(2): 91-99). In summary, female sexual dissatisfaction and dysfunction are highly prevalent and constitute a major health problem, despite its recognition as such and efforts to determine its etiology and to find effective treatments.

Female Sexual Disorders (FSD, herein used interchangeably with female sexual dysfunction) are defined in The Diagnostic and Statistical Manual of Mental Disorders-5 (DSM-5. 5th ed., 2013) and include Sexual Interest/Arousal Disorder and Female Orgasmic Disorder, as well as Genitopelvic Pain/Penetration Disorder. Attempts to improve arousal in women utilizing the same pharmacologic agents (e.g., sildenafil, tadalafil, prostaglandins) that have been successfully utilized in men with erectile dysfunction (ED) have produced no statistically significant effects in women, even when selective subpopulations have been examined, with the possible exception of sildenafil. The two currently approved treatments for FSD are for desire disorders, are both centrally acting drugs, and neither has gained traction in the marketplace. No peripherally acting agents have been approved by regulatory agencies (e.g., US FDA) for augmentation or enhancement of sexual function in otherwise normal or intact women (from a sexual function perspective) or for the treatment of interest/arousal disorders or orgasmic disorders in women. The available treatments for other types of female sexual dysfunction vary according to age/hormonal status (pre- or postmenopausal) and the diagnosis, and include hormonal therapies (testosterone, estrogen, and estrogen modulators such as ospemifene), vaginal lubricants and moisturizers, cognitive therapy, etc. (Frank, J. E., Mistretta, P., and Will, J. (2008) Diagnosis and treatment of female sexual dysfunction, American Family Physician 77, 635-642). The management of female sexual dysfunction secondary to antidepressant therapy is particularly problematic as no treatment is approved for this purpose, there are no clinical guidelines recommending a course of treatment, and precious few of myriad examined pharmacological agents worked better than placebo in rigorous clinical trials (Luft, M. J., Dobson, E. T., Levine, A., Croarkin, P. E., and Strawn, J. R. (2021) Pharmacologic interventions for antidepressant-induced sexual dysfunction: a systematic review and network meta-analysis of trials using the Arizona sexual experience scale, CNS Spectrums, 1-10). This is so despite long-standing recognition of female sexual dysfunction induced by antidepressants as a significant health problem and a major impediment to patient compliance, even for the newest SSRI and SNRI classes of antidepressant medications (Demyttenaere, K., Van Ganse, E., Gregoire, J., Gaens, E., Mesters, P. (1998) Compliance in depressed patients treated with fluoxetine or amitriptyline. Belgian Compliance Study Group. International Clinical Psychopharmacology 13, 11-7; Olah, K. S. (2002) The use of fluoxetine (Prozac) in premenstrual syndrome: is the incidence of sexual dysfunction and anorgasmia acceptable?Journal of Obstetrics and Gynaecology 22, 81-83). Extrapolating results in males to possible treatments in females is problematic, as multiple treatments found effective in males have failed to show benefit in females (for example, Farnia, V., Hojatitabar, S., Shakeri, J., Rezaei, M., Yazdchi, K., Bajoghli, H., Holsboer-Trachsler, E., and Brand, S. (2015) Adjuvant Rosa Damascena has a Small Effect on SSRI-induced Sexual Dysfunction in Female Patients Suffering from MDD, Pharmacopsychiatry 48, 156-163). Consequently, female sexual dysfunction induced by antidepressant medications is an important condition of unmet need, which has proven intractable despite decades of effort, with no obvious solutions on the horizon or research programs whose clinical success may be predicted with any degree of confidence.

Yet another example of major unmet clinical need is female sexual dysfunction in cancer survivors and/or the iatrogenic sexual dysfunction caused by the treatment of cancer. A recent failure of a Phase II clinical trial evaluating a potential treatment of this group of patients is illustrative of the difficulty and non-obvious nature of finding effective therapies (Barton, D. L., et al. (2022) Randomized Controlled Phase II Evaluation of Two Dose Levels of Bupropion Versus Placebo for Sexual Desire in Female Cancer Survivors: NRG-CC004, Journal of Clinical Oncology 40, 324-334; Lustberg, M. B., Loprinzi, C. L., and Streicher, L. (2022) A Pill for Sexual Desire in Female Cancer Survivors: Too Good to Be True?, Journal of Clinical Oncology 40, 317-319) The endocannabinoid system (ECS) is a major neuromodulatory regulatory system found in the central nervous system and in select peripheral nerves and organs. The canonical ECS is composed of cannabinoid (CB1 and CB2) receptors, their endogenous ligands (endocannabinoids: anandamide (AEA) and 2-arachidonoylglycerol), proteins involved in the synthesis and breakdown of endocannabinoids, and the intracellular signaling pathways affected by cannabinoids. CB1 and CB2 receptors are G-protein-coupled receptors that serve as the primary site of action for endocannabinoids (Pertwee, R. G., et al. (2010) International Union of Basic and Clinical Pharmacology. LXXIX. Cannabinoid receptors and their ligands: beyond CB(1) and CB(2), Pharmacological Reviews 62, 588-631). The cannabinoid receptors differ in their distribution. CB1 receptors are found throughout the central nervous system and some peripheral tissues. Cannabinoid receptors in the CNS are found in the hypothalamus, hippocampus, amygdala, cerebral cortex, parts of the basal ganglia, and cerebellum. CB1 and CB2 receptors are presynaptic receptors that results in inhibition of neurotransmitter release when activated. CB1 receptors are located in the axon terminals of GABAergic, dopaminergic, adrenergic, glutamatergic, cholinergic and some serotonergic neurons, particularly in the primitive portions of the limbic system that control, among other things, sexual behavior. Peripherally, CB2 cannabinoid receptors are found in organs responsible for producing sex hormones. They have also been found in the ovaries, uterus, bladder, penile corpora and the testes, as well as in the skin. To date, the female vagina and clitoris have not been examined for or determined to have CB receptors.

Δ9-(delta-9)-tetrahydrocannabinol (THC) was the first cannabinoid identified from the Cannabis sativa plant and characterized in the 1960's by Mechoulam and colleagues. It is a potent sedative-hypnotic. Subsequently, over a hundred cannabinoids, including cannabidiol (CBD), a non-psychoactive cannabinoid, have been identified. Cannabidiol has been attributed to have many potential pharmacological benefits in the management of pain, inflammation, irritable bowel syndrome, and infantile seizure disorders, among others. It recently received FDA approval for the treatment of Dravet syndrome and Lennox-Gastaut syndrome (Davinsky, O. et al. (2017) Trial of Cannabidiol for Drug-Resistant Seizures in the Dravet Syndrome, New England Journal of Medicine 376, 2011-2020; (2018) Cannabidiol in patients with seizures associated with Lennox-Gastaut syndrome (GWPCARE4): a randomised, double-blind, placebo-controlled phase 3 trial, Lancet 391, 1085-1096). The discovery and study of the potential benefits of CBD and other cannabinoids for human health, as well as the study of the ECS, has led to the expanded working definition of the endocannabinoid system, which now is argued to include multiple non-classical components such as PPAR-α, GPR18, GPR55, and GPR119, ion channels, and other receptors, enzymes, transporters, etc., and the non-classical endocannabinoids N-palmitoylethanolamine (PEA) and N-oleoylethanolamine (OEA), and their homologues and analogues, collectively dubbed the endocannabinoid-like substances (Lowe, H., Toyang, N., Steele, B., Bryant, J., and Ngwa, W. (2021) The Endocannabinoid System: A Potential Target for the Treatment of Various Diseases, International Journal of Molecular Sciences 22, 9472). The ECS and its signaling pathways as understood today are very complex.

Cannabidiolic acid (CBDA) is an acidic cannabinoid and a precursor to CBD, into which it is converted by decarboxylation. Compared with CBD and other decarboxylated cannabinoids it has not been studied extensively but it and other acidic cannabinoids are receiving increased attention (Formato, M., et al. (2020) (−)-Cannabidiolic Acid, a Still Overlooked Bioactive Compound: An Introductory Review and Preliminary Research, Molecules 25, 2638). One reason for the comparative neglect of CBDA is the perception of its chemical instability, which is an obvious potential impediment to its development given that the conversion of CBDA to CBD is a thermal process. Nonetheless, CBDA has sufficient stability at ambient temperature and it may be possible to stabilize it through formulation and by other means. One approach to the improvement of chemical stability of CBDA is the formation of its carboxylic ester derivatives, such as cannabidiolic acid methyl ester (CBDA-ME) HU-580.

The properties of CBDA are different from those of CBD, it cannot be considered a CBD analogue. First, the physicochemical properties of CBDA are vastly different from CBD. The pKₐ of CBDA has not been reported but may be estimated to be approximately 3.4 by comparison to olivetolic acid, meaning that unlike CBD it is completely ionized at physiological pH. Consequently, relative to CBD, CBDA is more water soluble, has lower LogP (LogD), has a different polar surface area value and electrostatic surface, a different geometry and number of hydrogen bond acceptors and donors, etc. High lipophilicity, as approximated by LogP (LogD) values, appears to be an important parameter for interactions with CB1 and CB2 receptors (Morales, P., Hurst, D. P., and Reggio, P. H. (2017) Molecular Targets of the Phytocannabinoids: A Complex Picture, In Phytocannabinoids: Unraveling the Complex Chemistry and Pharmacology of Cannabis sativa (Kinghorn, A. D., Falk, H., Gibbons, S., and Kobayashi, J. i., Eds.), pp 103-131, Springer International). The different physicochemical properties may be expected to translate into major differences in permeability, protein binding, and other pharmacologically relevant properties. Based on the available data, which is far from complete, CBDA and CBD have overlapping yet distinct biochemical profiles. For example, CBDA appears to be an inhibitor of cyclooxygenase isoforms (there is some controversy whether CBDA is selective for COX-2 or COX-1) whereas CBD had much weaker or no activity against this enzyme. Due to the known activity of CBDA against COX enzymes, CBDA was included in topical compositions for reduction of pain and post traumatic inflammation and/or arthritis/osteoarthritis (OA) in the deep tissues of joints and muscles (U.S. patent Ser. No. 10/918,686 B2, Oleo gel composition and delivery system with active compounds from Cannabis sativa and mentha arvensis for reduction of inflammation and pain in deep tissues). Furthermore, in cell-based assays, CBDA and CBD were found to have qualitatively similar effects on some receptors yet divergent effects on other signaling pathways (Navarro, G., et al. (2020) Pharmacological data of cannabidiol- and cannabigerol-type phytocannabinoids acting on cannabinoid CB1, CB2 and CB1/CB2 heteromer receptors, Pharmacological Research 159, 104940; Zagzoog, A., et al. (2020) In vitro and in vivo pharmacological activity of minor cannabinoids isolated from Cannabis sativa, Scientific Reports 10, 20405). Similarly, the known biochemical and pharmacological properties of other acidic cannabinoids overlap to some extent yet are distinct both from other acidic cannabinoids and from the decarboxylated cannabinoids, whereas based on the known properties of HU-580 it has stronger activity than CBDA at some of the receptors of interest. Therefore, with the present state of knowledge of cannabinoid and acidic cannabinoid biochemistry and pharmacology, it is impossible to produce confident predictions of the pharmacological effect of an untested cannabinoid or acidic cannabinoid in a target tissue by analogy to a previously tested compound or by extrapolating from one condition to another.

The role of the hypothalamic pituitary axis on female sex hormones and thus female sexual function has been long established. The neurohormonal aspects of male and female sexual desire or interest are driven by androgens. The endocannabinoid system appears to be inhibitory to sexual responses in animals. Levels of the endocannabinoids anandamide (AEA) and 2-arachidonoylglycerol (2-AG) are lowered in response to sexual stimulation and changes in their plasma concentrations are significantly inversely correlated with indices of sexual arousal (Klein, C., Hill, M. N., Chang, S. C., Hillard, C. J., and Gorzalka, B. B. (2012) Circulating endocannabinoid concentrations and sexual arousal in women, Journal of Sexual Medicine 9, 1588-1601). Delta-9 tetrahydrocannabinol (THC) appears to blunt the activation of hormones that modulate female sexual responses in animal and human studies. However, exogenous THC, in the presence of an intact hormonal axis, produces female rat lordosis (a sexual receptivity posture) at low doses. This is believed to be an entirely central nervous system effect and not a peripheral response. While CB1 and CB2 receptors for endocannabinoids exist peripherally, the actions of THC on sexual function are believed to be central in action on the dopaminergic and serotonergic pathways of the limbic system (hypothalamic ventral tegmental area and nucleus accumbens). Pharmacologic doses of Δ9-tetrahydrocannabinol appear to augment these serotonergic and dopaminergic pathways. (Lynn, B. K., López, J. D., Miller, C., Thompson, J., and Campian, E. C. (2019) The Relationship between Marijuana Use Prior to Sex and Sexual Function in Women, Sexual Medicine 7,192-197).

In contrast, sexual arousal is a peripheral genital process. Female and male sexual arousal, as evidenced by clitoral engorgement and vaginal lubrication, or penile erection, respectively, are regulated by the tone of the smooth muscle of the clitoris and vagina and by the tone of the smooth muscle of the corpora cavernosa and corpus spongiosum. CB1 and CB2 receptors have been identified in the cavernosal endothelium and smooth muscle of male primates and humans (Gratzke, C., Christ, G. J., Stief, C. G., Andersson, K. E., and Hedlund, P. (2010) Localization and function of cannabinoid receptors in the corpus cavernosum: basis for modulation of nitric oxide synthase nerve activity, European Urology 57, 342-348). Sexual arousal in males may be related to nitrergic pathways in the corpus cavernosum. However, despite identification of CB2 receptors in ovaries and endometrial tissue, there have been no studies to confirm the presence of vaginal or clitoral CB receptors in animal or human tissue and evidence to support a role of the endocannabinoid system to effect arousal through a peripheral pharmacologic action on this smooth muscle.

To date, only one study of a direct pharmacologic effect of an individual cannabinoid on vaginal or clitoral smooth muscle function has been disclosed (WO2021091908A1 Frid, M., and Padma-Nathan, H. Peripherally acting cannabidiol (CBD)-containing compositions and uses thereof for enhancing female sexual function or treating female sexual disorders), using the gold-standard experimental approach to demonstrate a pharmacologic impact on smooth muscle and its related endothelium, which are performed in isolated tissue organ baths, examining the dose or concentration related relaxations responses (lowered isometric tension) in precontracted smooth muscle tissue (Munarriz, R., Kim, S. W., Kim, N. N., Traish, A., and Goldstein, I. (2003) A review of the physiology and pharmacology of peripheral (vaginal and clitoral) female genital arousal in the animal model, Journal of Urology 170, S40-44). In that study, a statistically-significant relaxation by CBD of vaginal smooth muscle was demonstrated. On the other hand, the mechanism by which CBD relaxes vaginal smooth muscle is unknown, nor is it known which receptor(s) it interacts with to effect the smooth muscle relaxation and whether or not those receptors are part of the canonical or even the expended endocannabinoid system. The pharmacology of CBD, arguably the most studied of phytocannabinoids, is complex, may be influenced by other active substances present, for example THC, and extends beyond CB1 and CB2 receptors. CBD is now known to interact with a variety of receptors, including multiple TRP receptors, serotonin receptors, PPARγ, GPR55, and others, and may well involve simultaneous interactions with two or more receptors and/or enzymes (Vitale, R. M., lannotti, F. A., and Amodeo, P. (2021) The (Poly)Pharmacology of Cannabidiol in Neurological and Neuropsychiatric Disorders: Molecular Mechanisms and Targets, International Journal of Molecular Sciences 22, 4876); CBD is suggested as potential treatment for a wide variety of conditions (Britch, S. C., Babalonis, S., and Walsh, S. L. (2021) Cannabidiol: pharmacology and therapeutic targets, Psychopharmacology 238, 9-28). Further complicating matters is the apparent ability of CBD to effect signaling despite its low affinity for the target receptors; CB1 and CB2 are prime examples of this dichotomy. Nevertheless, it was found that topical administration of liposomal CBD temporarily reversed the SSRI-induced female anorgasmia (WO2021188388A1 Frid, M., Padma-Nathan, H., Segil, H., and DeMena, N. C. Use of cannabidiol in treating anti-depressant-induced female sexual dysfunction).

Thus, there remains a need to provide novel composition and methods for improving female sexual function and treating or ameliorating certain sexual disorders in women. Despite the current impossibility of mechanistic analysis or prediction of pharmacological effect of CBD and other cannabinoids in specific tissues using specific delivery methods, given the available efficacy signals, there is a need to provide more effective than CBD in enhancing female sexual function, reducing female sexual dissatisfaction, and/or treating or ameliorating certain sexual disorders in women.

### SUMMARY OF THE INVENTION

The present disclosure provides compositions and methods of using thereof for enhancing female sexual function, or reducing female sexual dissatisfaction, or treating female sexual dysfunction containing cannabidiolic acid (CBDA). Unexpectedly, it was found that CBDA was not only effective but significantly more effective than CBD at relaxing a precontracted rat vaginal smooth muscle in a dose-dependent manner.

In some embodiments, the present disclosure provides peripherally acting compositions comprising CBDA and methods of using thereof for improving female sexual satisfaction and/or reducing female sexual dissatisfaction by improving sexual function of a subject whereby the application of CBDA-containing compositions a period of time prior to a sexual activity results in increase of subjective measures of desire, arousal, and/or lubrication, and/or orgasm, or reducing or eliminating pain during penetrative sex (dyspareunia).

In some embodiments, the present disclosure provides peripherally acting compositions comprising CBDA and methods of using thereof for treating primary female sexual dysfunction. In some embodiments, the primary female sexual dysfunction (PFSD) is related to arousal and/or lubrication, including but not limited to difficulty in achieving and maintaining engorgement, and difficulty in achieving and maintaining lubrication. In some embodiments, PFSD is related to orgasm, including but not limited to, difficulty achieving orgasm, low orgasm intensity, or anorgasmia. In some embodiments, PFSD is related to lack of interest or desire. In some embodiments, PFSD is related to pain during penetrative sex (dyspareunia). In general, the compositions of the invention are applied as a lotion, a cream, a gel, or a similar formulation in the amount and for a period of time prior to a sexual activity such that sexual function of the subject is enhanced and/or the PFSD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm”, “reaching orgasm more often”; “greater ease of achieving orgasm”; “being more satisfied”; “higher level of sexual desire”; and/or “reduction in pain during sexual activity”.

In some embodiments, the present disclosure provides peripherally acting compositions comprising CBDA and methods of using thereof treating female sexual dysfunction secondary to treatment with antidepressants (antidepressant-induced sexual dysfunction, AISD). In some embodiments, the female sexual dysfunction is induced by SSRIs, SNRIs, tricyclic, or tetracyclic antidepressants. In some embodiments, AISD is related to arousal and/or lubrication, including but not limited to difficulty in achieving and maintaining engorgement, and difficulty in achieving and maintaining lubrication. In some embodiments, AISD is related to orgasm, including but not limited to, difficulty achieving orgasm, low orgasm intensity, or anorgasmia. In some embodiments, AISD is related to lack of interest or desire. In general, the compositions of the invention are administered as a lotion, a cream, a gel, or a similar formulation in the amount and for a period of time prior to a sexual activity such that sexual function of the subject is enhanced and/or AISD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm” if anorgasmic after starting antidepressant therapy, “reaching orgasm more often”; “greater ease of achieving orgasm”; “being more satisfied”; “higher level of sexual desire”; and “reduction in pain during sexual activity”. In other embodiments, the compositions of the invention are administered as a lotion, a cream, a gel, or a similar formulation in the amount and for a period of time prior to a sexual activity such that sexual function of the subject is enhanced and/or the disorder is ameliorated during the sexual activity, such that some proportion of sexual function of the subject lost due to antidepressant-induced sexual dysfunction is recovered to levels experienced prior to starting antidepressant therapy, or is enhanced, if the subject was not sexually active prior to starting antidepressant therapy, said improvements determined using questionnaires derived from the Female Sexual Function Index questionnaire (FSFI) or the Arizona Sexual Experiences Scale (ASEX) questionnaire, or equivalent.

In some embodiments, the present disclosure provides peripherally acting CBDA-containing compositions and methods of using thereof for treating female sexual dysfunction in cancer survivors and/or the sexual dysfunction secondary to the treatment of cancer. In some embodiments, SD is related to arousal and/or lubrication, including but not limited to difficulty in achieving and maintaining engorgement, and difficulty in achieving and maintaining lubrication. In some embodiments, SD is related to orgasm, including but not limited to, difficulty achieving orgasm, low orgasm intensity, or anorgasmia. In some embodiments, SD is related to lack of interest or desire. In some embodiments, SD is related to pain during penetrative sex (dyspareunia). In general, the compositions of the invention are administered as a lotion, a cream, a gel, or a similar formulation in the amount and for a period of time prior to a sexual activity such that sexual function of the subject is enhanced and/or SD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm” if anorgasmic after cancer therapy, “reaching orgasm more often”; “greater ease of achieving orgasm”; “being more satisfied”; “higher level of sexual desire”; and “reduction in pain during sexual activity”. In other embodiments, the compositions of the invention are administered as a lotion, a cream, a gel, or a similar formulation in the amount and for a period of time prior to a sexual activity such that sexual function of the subject is enhanced and/or the disorder is ameliorated during the sexual activity, such that some proportion of sexual function of the subject lost due to the effects of cancer, the effects of treatment, or SD that persists after the cessation of treatment, is recovered to levels experienced prior to the onset of cancer, or is enhanced, if the subject was not sexually active prior to starting antidepressant therapy, said improvements determined using questionnaires derived from the Female Sexual Function Index questionnaire (FSFI) or the Arizona Sexual Experiences Scale (ASEX) questionnaire, or equivalent.

In some embodiments, the subjects are premenopausal women, while in other embodiments the subjects are post-menopausal women. In some embodiments, the subjects are women who have given birth, while in other embodiments the subjects have never given birth.

In general, the methods of the invention employ a topical composition comprising CBDA and, optionally, one or more additional cannabinoids, and, optionally, one or more non-cannabinoid pharmacological agents. In the methods of the invention, such a composition is applied topically to a female subject's genital (arousal) area(s), to surface with absorptive mucosa, such as, for example, the vulva, the introitus, the labia minora, the clitoris, the vaginal vault, and/or intravaginally.

In general, the compositions of the invention are administered as a lotion, a cream, a gel, a suppository, a lube, or a similar formulation in the amount and for a period of time prior to a sexual activity. In some embodiments, the subject is treated with a composition comprising CBDA and, optionally, other cannabinoid and/or non-cannabinoid ingredients, continuously for a period of 3-6 months, and exhibits improvement in one or more parameters of sexual function, as measured by FSFI, for example, in sexual interest/arousal domains is by 1.5-2 points, and by 1.5-2 points in the orgasm domain of the FSFI.

A more detailed description of the invention follows.

### DETAILED DESCRIPTION OF THE DRAWINGS

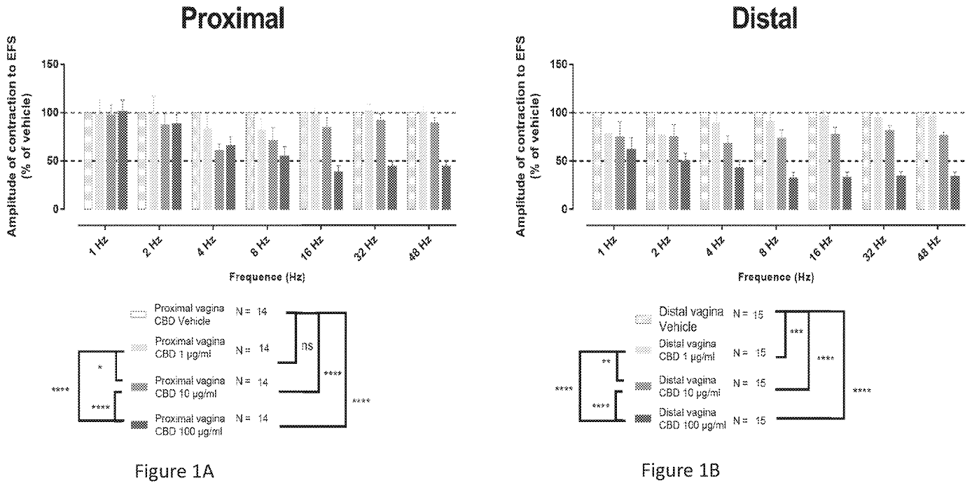

FIG. 1A demonstrates that CBD has a statistically significant, dose-dependent pharmacological relaxant effect on rat proximal vaginal smooth muscle tissue.

FIG. 1B demonstrates that CBD has a statistically significant, dose-dependent pharmacological relaxant effect on rat distal vaginal smooth muscle tissue.

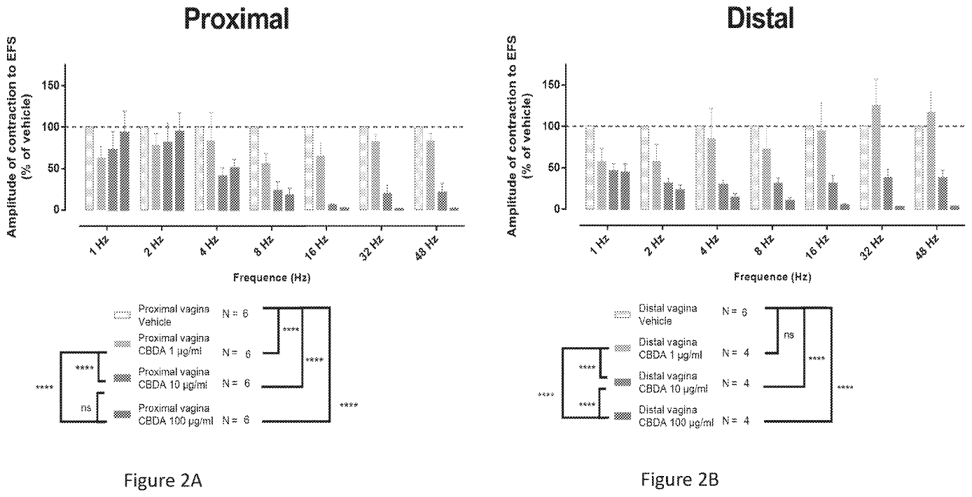

FIG. 2A demonstrates that CBDA has an unexpected, statistically significant, dose-dependent pharmacological relaxant effect on rat proximal vaginal smooth muscle tissue.

FIG. 2B demonstrates that CBDA has an unexpected, statistically significant, dose-dependent pharmacological relaxant effect on rat distal vaginal smooth muscle tissue.

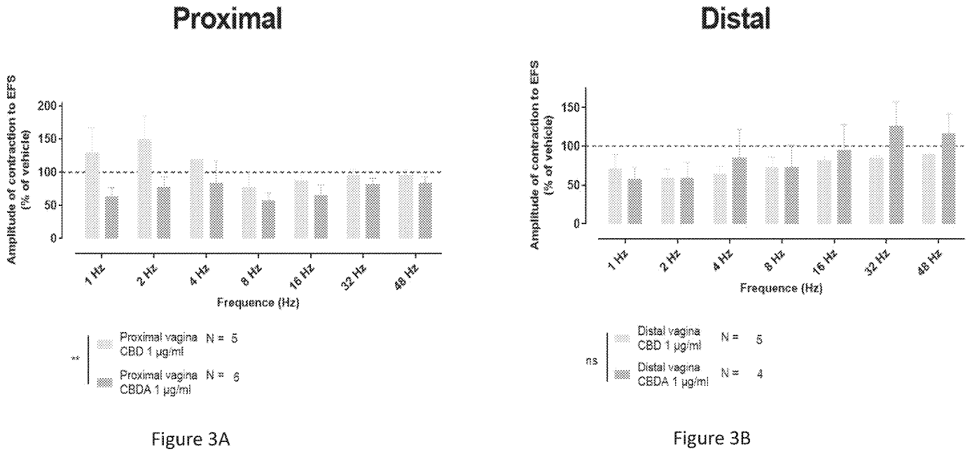

FIG. 3A shows a comparison of pharmacological relaxant effect of 1 μg/mL of CBD and CBDA on rat proximal vaginal smooth muscle tissue.

FIG. 3B shows a comparison of pharmacological relaxant effect of 1 μg/mL CBD and CBDA on rat distal vaginal smooth muscle tissue.

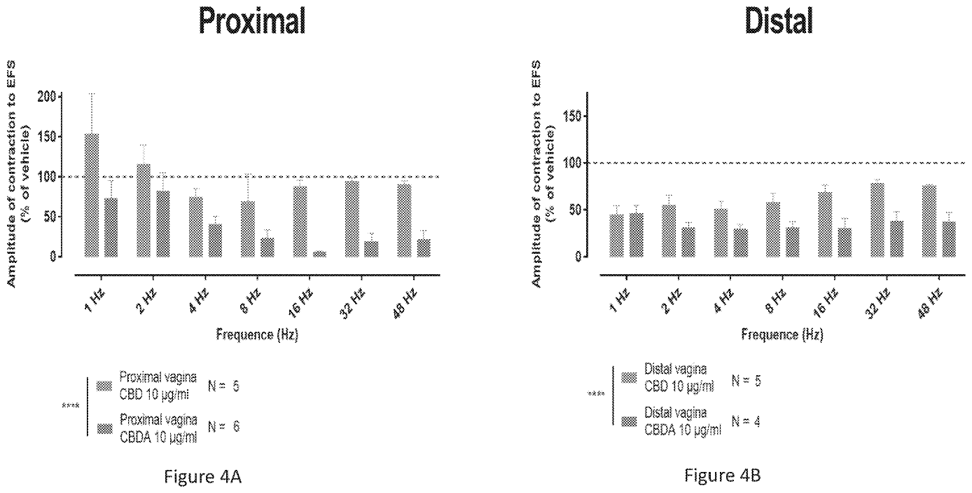

FIG. 4A shows a statistically significant difference in the pharmacological relaxant effect of 10 μg/mL of CBD and CBDA on rat proximal vaginal smooth muscle tissue.

FIG. 4B shows a statistically significant difference in the pharmacological relaxant effect of 10 μg/mL CBD and CBDA on rat distal vaginal smooth muscle tissue.

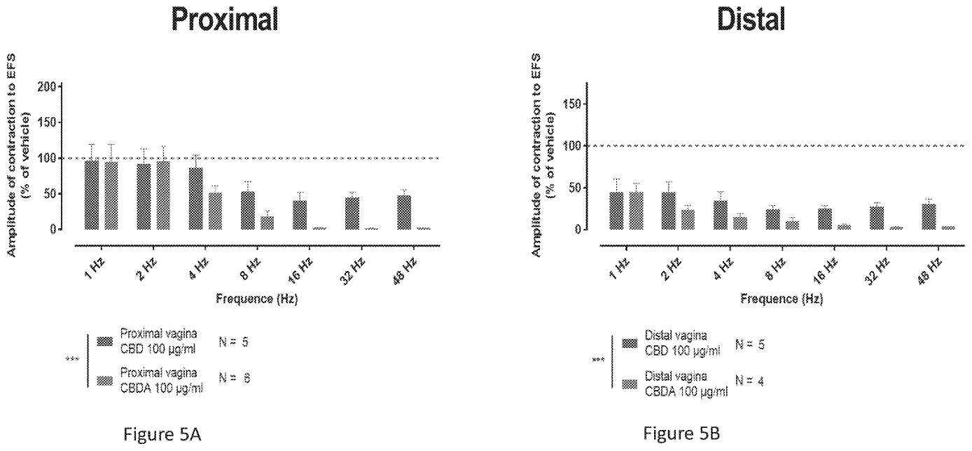

FIG. 5A shows a statistically significant difference in the pharmacological relaxant effect of 100 μg/mL of CBD and CBDA on rat proximal vaginal smooth muscle tissue.

FIG. 5B shows a statistically significant difference in the pharmacological relaxant effect of 100 μg/mL CBD and CBDA on rat distal vaginal smooth muscle tissue.

### DETAILED DESCRIPTION OF THE INVENTION

The present disclosure provides compositions and methods of using thereof for enhancing female sexual function, or reducing female sexual dissatisfaction, or treating female sexual dysfunction containing cannabidiolic acid (CBDA):

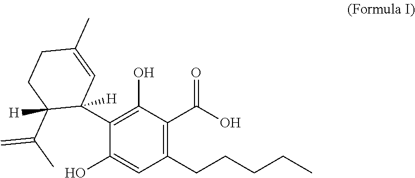 [SMILES: C=C(C)[C@@H]1CCC(C)=C[C@H]1c1c(O)cc(CCCCC)c(C(=O)O)c1O]

In the methods of the invention, such a composition is applied topically to a female subject's genital (arousal) area(s), to surface with absorptive mucosa, such as, for example, the introitus, the labia minora, the clitoris and the vaginal vault. In some embodiments, the composition comprising CBDA is applied intravaginally.

In some embodiments, the present disclosure provides peripherally acting compositions comprising CBDA and methods of using thereof for improving female sexual satisfaction and/or reducing female sexual dissatisfaction by improving sexual function of a subject whereby the application of CBDA-containing compositions a period of time prior to a sexual activity results in increase of subjective measures of desire, arousal, and/or lubrication, and/or orgasm, or reducing or eliminating pain during penetrative sex (dyspareunia); as used herein, dyspareunia may include pain caused entirely or in large part by vaginismus or lack of lubrication. The improvement(s) female sexual satisfaction and/or reduction in female sexual dissatisfaction may be as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm”, “reaching orgasm more often”; “greater ease of achieving orgasm”; “being more satisfied”; “higher level of sexual desire”; “lower level of anxiety prior to or during sexual activity”, and/or “reduction in pain during sexual activity”.

In some embodiments, the present disclosure provides peripherally acting compositions comprising CBDA and methods of using thereof for treating primary female sexual dysfunction. Herein, primary dysfunction refers to a lifelong condition, as distinct from a secondary or acquired dysfunction, which results from the effects of a disease or a disorder (e.g., a psychiatric disorder) or the effects of treatment(s) of a disease or a disorder, whether pharmacological, surgical, or other, which effects led to a decline or decrease in one or more aspects of sexual function from the previous level(s). In some embodiments, the primary female sexual dysfunction (PFSD) is related to arousal and/or lubrication, including but not limited to difficulty in achieving and maintaining engorgement, and difficulty in achieving and maintaining lubrication. In some embodiments, PFSD is related to orgasm, including but not limited to, difficulty achieving orgasm, low orgasm intensity, or anorgasmia. In some embodiments, PFSD is related to lack of interest or desire. In some embodiments, PFSD is related to pain during penetrative sex (dyspareunia); as used herein, dyspareunia may include pain caused entirely or in large part by vaginismus or lack of lubrication. In general, the compositions of the invention are administered (applied) in the amount and for a period of time prior to a sexual activity such that sexual function of the subject is enhanced and/or the PFSD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm”, “reaching orgasm more often”; “greater ease of achieving orgasm”; “being more satisfied”; “higher level of sexual desire”; and/or “reduction in pain during sexual activity”. In other embodiments, the invention features treating a female sexual disorder such as, for example, Sexual Interest/Arousal Disorder (SIAD) and Female Orgasmic Disorder.

In some embodiments, the present disclosure provides peripherally acting compositions comprising CBDA and methods of using thereof treating female sexual dysfunction secondary to treatment with antidepressants (antidepressant-induced sexual dysfunction, AISD). In some embodiments, the female sexual dysfunction is induced by SSRIs, SNRIs, tricyclic, or tetracyclic antidepressants. In some embodiments, AISD is related to arousal and/or lubrication, including but not limited to difficulty in achieving and maintaining engorgement, and difficulty in achieving and maintaining lubrication. In some embodiments, AISD is related to orgasm, including but not limited to, difficulty achieving orgasm, low orgasm intensity, or anorgasmia. In some embodiments, AISD is related to lack of interest or desire. In general, the compositions of the invention are administered in the amount and for a period of time prior to a sexual activity such that sexual function of the subject is enhanced and/or AISD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm” if anorgasmic after starting antidepressant therapy, “reaching orgasm more often”; “greater ease of achieving orgasm”; “being more satisfied”; “higher level of sexual desire”; and “reduction in pain during sexual activity”. In other embodiments, the compositions of the invention are administered as a lotion, a cream, a gel, or a similar formulation in the amount and for a period of time prior to a sexual activity such that sexual function of the subject is enhanced and/or the disorder is ameliorated during the sexual activity, such that some proportion of sexual function of the subject lost due to antidepressant-induced sexual dysfunction is recovered to levels experienced prior to starting antidepressant therapy, or is enhanced, if the subject was not sexually active prior to starting antidepressant therapy, said improvements determined using questionnaires derived from the Female Sexual Function Index questionnaire (FSFI) or the Arizona Sexual Experiences Scale (ASEX) questionnaire, or equivalent.

The present disclosure provides peripherally acting cannabidiolic acid (CBDA)-containing compositions and methods of using thereof for treating female sexual dysfunction secondary to treatment with antidepressants (antidepressant-induced sexual dysfunction), whether or not the antidepressants are used in the treatment of MDD or other depressive spectrum disorders (persistent depressive disorder or dysthymia, melancholic depression, etc.), anxiety disorders such as generalized anxiety disorder (GAD) and social anxiety disorder (SAD), attention-deficit hyperactivity disorder (ADHD), obsessive-compulsive disorder (OCD), bulimia nervosa (bulimia), panic disorder, premenstrual dysphoric disorder, menopause-associated vasomotor symptoms, fibromyalgia, neuropathic pain, post-traumatic stress disorder (PTSD), diabetic peripheral neuropathy (DPN), chemotherapy-induced neuropathy, or for the treatment of another indication.

The female sexual dysfunction can be secondary to treatment with anti-depressants such as, e.g., selective serotonin reuptake inhibitors (SSRIs), serotonin norepinephrine reuptake inhibitors (SNRIs), tricyclic antidepressants, and tetracyclic antidepressants. See US Patent Appln. Pub. No. 2021-0283069. Accordingly, in some embodiments, the female sexual dysfunction is secondary to treatment with SSRIs or SNRIs. In some embodiments, the female sexual dysfunction is secondary to treatment with tricyclic or tetracyclic antidepressants. In some particular embodiments, the female sexual dysfunction is secondary to treatment with sertraline, citalopram, fluoxetine, escitalopram, paroxetine, fluvoxamine, duloxetine, venlafaxine, and desvenlafaxine. In other particular embodiments, the female sexual dysfunction is secondary to treatment with sertraline, citalopram, fluoxetine, escitalopram, paroxetine, and fluvoxamine. In other particular embodiments, the female sexual dysfunction is secondary to treatment with sertraline, citalopram, and fluoxetine. In other particular embodiments, the female sexual dysfunction is secondary to treatment with duloxetine, venlafaxine, desvenlafaxine, milnacipran, and levomilnacipran. In other particular embodiments, the female sexual dysfunction is secondary to treatment with clomipramine, trimipramine, amitriptyline, desipramine, imipramine, lofepramine, doxepin, nortriptyline, amoxapine, and protriptyline. In still other embodiments, the female sexual dysfunction is secondary to treatment with maprotiline and mirtazapine. In general, sexual dysfunction induced by any anti-depressant of the SSRI, SNRI, tricyclic, or tetracyclic classes is susceptible to the treatment/amelioration with the methods of the present invention. In certain embodiments, the subject was previously treated with an anti-depressant that induced sexual dysfunction and has persistent sexual dysfunction despite discontinuation of the anti-depressant therapy.

In some embodiments, the present disclosure provides peripherally acting CBDA-containing compositions and methods of using thereof for treating female sexual dysfunction in cancer survivors and/or the sexual dysfunction secondary to the disease of cancer or the treatment of cancer. In some embodiments, SD is related to arousal and/or lubrication, including but not limited to difficulty in achieving and maintaining engorgement, and difficulty in achieving and maintaining lubrication. In some embodiments, SD is related to orgasm, including but not limited to, difficulty achieving orgasm, low orgasm intensity, or anorgasmia. In some embodiments, SD is related to lack of interest or desire. In some embodiments, SD is related to pain during penetrative sex (dyspareunia); as used herein, dyspareunia may include pain caused entirely or in large part by vaginismus or lack of lubrication. In general, the compositions of the invention are administered in the amount and for a period of time prior to a sexual activity such that sexual function of the subject is enhanced and/or SD is ameliorated during the sexual activity, for example, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm” if anorgasmic after starting antidepressant therapy, “reaching orgasm more often”; “greater ease of achieving orgasm”; “being more satisfied”; “higher level of sexual desire”; and “reduction in pain during sexual activity”. In other embodiments, the compositions of the invention are administered in the amount and for a period of time prior to a sexual activity such that sexual function of the subject is enhanced and/or the disorder is ameliorated during the sexual activity, such that some proportion of sexual function of the subject lost due to the effects of cancer, the effects of treatment, or SD that persists after the cessation of treatment, is recovered to levels experienced prior to the onset of cancer, or is enhanced, if the subject was not sexually active prior to starting antidepressant therapy, said improvements determined using questionnaires derived from the Female Sexual Function Index questionnaire (FSFI) or the Arizona Sexual Experiences Scale (ASEX) questionnaire, or equivalent.

With respect to treatment of sexual disorders, it is expected that the subject, upon having been treated with the CBDA-containing composition for 3-6 months, exhibits improvement in one or more parameters of sexual function, as measured by FSFI, for example, in sexual interest/arousal domains is by 1.5-2 points, and by 1.5-2 points in the orgasm domain of the FSFI.

In some embodiments, the subjects are premenopausal women, while in other embodiments the subjects are post-menopausal women. In some embodiments, the subjects are women who have given birth, while in other embodiments the subjects have never given birth.

In general, the compositions of the invention can be administered (applied) 1-60 min prior to sexual activity, or 10-60 min, or 5-30 minutes prior to sexual activity, or 10-30 min, preferably, 5-40 min, more preferably 15-20 min, or 15-40 min. In some embodiments, the off-set time following the application of the CBDA-containing composition is 0.5-5 hours, preferably, 1-3 hours, more preferably 1-2 hours.

In general, the total amount of CBDA per application is from 1 mg to 1,000 mg, and may be approximately 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, 50 mg of CBDA, preferably 10-100 mg of CBDA, more preferably, 20-40 mg of CBDA, most preferably 20 mg. Alternatively, the concentration of CBDA in a formulation may be 1 mg/g, 2 mg/g, 3 mg/g, 4 mg/g, 5 mg/g, 6 mg/g, 7 mg/g, 8 mg/g, 9 mg/g, 10 mg/g, 11 mg/g, 12 mg/g, 13 mg/g, 14 mg/g, 15 mg/g, 16 mg/g, 17 mg/g, 18 mg/g, 19 mg/g, 20 mg/g, 21 mg/g, 22 mg/g, 23 mg/g, 24 mg/g, 25 mg/g, 26 mg/g, 27 mg/g, 28 mg/g, 29 mg/g, 30 mg/g, 31 mg/g, 32 mg/g, 33 mg/g, 34 mg/g, 35 mg/g, 36 mg/g, 37 mg/g, 38 mg/g, 39 mg/g, or 40 mg/g.

The CBDA-containing composition may be applied multiple-times and the total dose may be subject-specific.

In some embodiments, the topical compositions are provided in the form of a solution in a plant oil or an oily vehicle known in the art as suitable for topical and transdermal administration. In some embodiments, the topical compositions are provided in the form of a serum, a gel, a lotion, a cream, an ointment, a balm, or a paste, the general composition of each of such dosage forms is known to persons skilled in the art. In some embodiments, the topical compositions are vaginal lubricants, either aqueous (water-based) or silicone-based, the general composition of such dosage forms is known to persons skilled in the art. In some embodiments, the compositions are provided in the form of a vaginal suppository.

In some embodiments, the compositions are provided in the form of a lotion, containing CBDA-loaded liposomes, the general production techniques of liposomes are known to those skilled in the arts, including the thin layer hydration method, evaporation of water-immiscible solvent (e.g. diethyl ether), or via freeze drying or lyophilization, or by dialysis through a size exclusion membrane after hydration, and including preparation of dehydrated liposomes (pro-liposomes or proliposomes). In some embodiments, the compositions are provided in the form of a suspension, whether transparent or opaque, containing CBDA-loaded liposomes. The mean particle size of the liposomal vesicles may be ≤100 nm, 101-500 nm, 501-1000 nm, and >1 μm, the distribution of these sizes may be of different dispersity (degree of non-uniformity of a size distribution of particles). Liposomes may be unilamellar, multilamellar, or a mixture of unilamellar and multilamellar liposomes. In some embodiments, CBDA-containing composition also comprises liposomes that comprise hydrogenated phosphatidylcholine (HSPC), lactic acid, propylene glycol, polyacrylate crosspolymer-6, and water or aqueous buffer, and wherein the liposomes are provided in a homogeneous suspension.

In some embodiments, the compositions are provided in the form of an emulsion containing CBDA, for example an oil-in-water (O/W) emulsion, or a double emulsion of the oil-in-water-in oil (O/W/O) type, or a double emulsion of the water-in-oil-in-water (W/O/W) type, the general production techniques for production of emulsions are known to those skilled in the arts, such as by means of spontaneous emulsification, phase inversion, solvent displacement, microfluidization, high shear mixing, high pressure homogenization, ultrasonic homogenization (colloquially, sonication), etc. The mean particle size of the dispersed phase may be ≤100 nm, 101-500 nm, 501-1000 nm, and >1 μm, the distribution of these sizes may be of different dispersity (degree of non-uniformity of a size distribution of particles).

In some embodiments, the topical compositions containing CBDA may be occlusive or non-occlusive.

In some embodiments, the topical compositions comprising CBDA are condom compatible, meaning that these compositions do not degrade or compromise barrier properties of natural rubber latex, and/or synthetic latex, and/or polyisoprene, and/or polyurethane male or female condoms. In some embodiments, a male condom is manufactured pre-coated with a composition comprising CBDA. In some embodiments, a female condom is manufactured pre-coated with a composition comprising CBDA suitable for intravaginal administration.

In some embodiments, the compositions comprising CBDA are provided in the form of a vaginal suppository. The suppository base may be hydrophobic, for example, cocoa butter, hard fat consisting of mixture(s) of mono-, di- and (mostly) triglyceride esters of saturated C10-C18 fatty acids, or mixtures of hard fat and ethoxylated fatty alcohols, and/or hard fat and glycerin monoester of long-chain saturated or unsaturated fatty acids such as glyceryl ricinoleate. In general, the melting ranges of the suppository bases are 30-60° C., 30-50° C., or 30-40° C., or 31-33° C., 32-34° C., 32-35° C., 33-36° C. In some embodiments, the suppository base may be hydrophilic, for example, a polyethylene glycol (PEG) of different molecular weights, for example, PEG 3350, or a mixture of PEGs of different molecular weights, or a mixture of PEGs and lubricating and solubilizing ingredients, with the melting ranges of 30-60° C., 30-50° C., or 30-40° C. The mass of the vaginal suppository may be in the range of 2.0-5.0 grams, or 2.5-4.0 grams, or 2.5-3.5 grams, or approximately 3.0 grams.

As used herein, cannabidiolic acid (CBDA) refers to the isomer (1′R,2′R)-2,6-dihydroxy-5′-methyl-4-pentyl-2′-(prop-1-en-2-yl)-1′,2′,3′,4′-tetrahydro[1,1′-biphenyl]-3-carboxylic acid produced, in whatever quantity, by plants Cannabis sativa and Cannabis indica, which naturally contain different amounts of CBDA, and to synthetic CBDA, which may be manufactured by isolation from Cannabis plants and chemovars thereof, by using yeast or other means utilizing biotechnology, by chemical synthesis, by combination of these methods, or by any other means. CBDA may be an amorphous solid, a crystalline solid of one or more than one polymorphs, or a mixture of amorphous and crystalline solids.

In some embodiments, the thermal stability (stability to decarboxylation) of CBDA may be improved by formation of a carboxylic acid salt. Salts of CBDA according to the present invention are preferably pharmaceutically acceptable salts, such as those containing counterions present in drug products listed in the US FDA Orange Book database. They can be formed in a customary manner, for example, by reacting CBDA with a suitable base or by other means known by persons skilled in the art. Suitable cationic counterions are in particular the ions of the alkali metals, preferably lithium, sodium and potassium, of the alkaline earth metals, preferably calcium, magnesium and barium, and of the transition metals, preferably manganese, copper, silver, zinc and iron, and also ammonium (NH4+) and substituted ammonium in which one to four of the hydrogen atoms are replaced by C1-C4-alkyl, C1-C4-hydroxyalkyl, C1-C4-alkoxy, C1-C4-alkoxy-C1-C4-alkyl, hydroxy-C1-C4-alkoxy-C1-C4-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore the cations of 1,4-piperazine, meglumine, benzathine and lysine. The stability to decarboxylation may be determined by means known in the art, such as by the comparison of onset of decomposition by differential scanning calorimetry (DSC), whereby the DSC onset of decomposition of the salt is 5° C., or 10° C., or 15° C., or 16 or more ° C. higher than for CBDA, as measured on substances of approximately the same purity (by HPLC) and with the same thermal ramp program. Alternatively, the stability to decarboxylation of CBDA and its salt may be compared by measuring the decrease of CBDA or its salt concentration in a composition over time stored at constant temperature, for example at ambient temperature, at 25° C., 30° C., 40° C., 50° C., 60° C., or another temperature, said concentrations determined quantitatively by HPLC relative to external or internal standards, or by other comparable means known in the art, such that after a time period of storage the salt is less degraded than CBDA by 5%, 10%, 15%, 20%, 25%, or higher, by comparison of the ratios of concentrations at time t to the initial concentrations at time t0 ([CBDA]t/[CBDA]t0 vs ([CBDA salt]t/[CBDA salt]t0.

In yet other embodiments, the composition comprising CBDA includes one or more carboxylated or decarboxylated phytocannabinoids, which are produced in whatever quantity, by plants Cannabis sativa and Cannabis indica, which naturally contain different amounts of the individual cannabinoids (Elsohly, M. A. and D. Slade (2005). “Chemical constituents of marijuana: the complex mixture of natural cannabinoids.” Life Sciences 78(5): 539-548), and to synthetic analogues of phytocannabinoids, which compounds may be manufactured by isolation from Cannabis plants and chemovars thereof, by using yeast or other means utilizing biotechnology, by chemical synthesis, by combination of these methods, or by any other means. The terms “cannabinoid” or “cannabinoids” refer to decarboxylated compounds or oxidized decarboxylated compounds having logP or clogP or 4, wherein logP is an n-octanol/water partition coefficient obtained experimentally or calculated (clogP) by methods known to those skilled in the art. The terms “cannabinoid” or “cannabinoids” refer, therefore, for example, to (−)-trans-Δ⁹-tetrahydrocannabinol (Δ⁹-THC or THC), Δ⁸-tetrahydrocannabinol (Δ⁸-THC), (−)-trans-cannabidiol (CBD), cannabinol (CBN), cannabichromene (CBC), cannabicyclol (CBL), cannabielsoin (CBE), cannabinoldiol, cannabitriol, cannabigerol (CBG), cannabifuran (CBF), and their homologues containing a propyl rather than a pentyl side chain, such as cannabidivarin (CBDV), cannabivarin (CBV or cannabivarol), tetrahydrocannabivarin (THCV or THV), cannabichromene propyl analogue, as well as nabilone (racemic mixture or mixture of individual enantiomers in whatever stereoisomeric excess or purity), levonantradol (CP 50,556-1), cannabilactone (AM-1714), cannabicyclohexanol ((C8)-CP 47,497), (C9)-CP 47,497, AM-2389, AM-4030, AM-4056, (−)-HU-210, (+)-HU-210, racemic HU-210 or a mixture of individual enantiomers in whatever stereoisomeric excess or purity, ajulemic acid (HU-239), HU-243, HU-308, HU-320, HU-331, HU-336, HU-345, 11-hydroxy-Δ⁹-tetrahydrocannabinol (11-OH-THC), 11-carboxy-Δ⁹-tetrahydrocannabinol (11-CO₂H-THC), 7-hydroxycannabidiol (7-OH-CBD), 7-carboxycannabidiol (7-OH-CBD), CP 55,940, JWH-133, AM-087, AM-356, AM-404, AM-678, AM-855, AM-905, AM-906, AM-919, AM-938, CP 47,497, (C6)-CP 47,497, (C7)-CP 47,497, CP 55,940, AMG-36, AMG-41, KM-233, JWH-051, JWH-102, JWH-056, JWH-057, JWH-065, JWH-103, JWH-133, JWH-139, JWH-142, JWH-143, JWH-161, JWH-186, JWH-187, JWH-188, JWH-190, JWH-191, JWH-215, JWH-216, JWH-217, JWH-224, JWH-225, JWH-226, JWH-227, JWH-229, JWH-230, JWH-233, JWH-247, JWH-254, JWH-256, JWH-277, JWH-278, JWH-298, JWH-299, JWH-300, JWH-301, JWH-310, JWH-336, JWH-338, JWH-339, JWH-340, JWH-341, JWH-349, JWH-350, JWH-352, JWH-353, JWH-354, JWH-355, JWH-356, JWH-357, JWH-358, JWH-359, JWH-360, JWH-361, JWH-362, 0-1871, and any combination of two or more of these compounds. Cannabinoids may be isolated from plants as mixtures of cannabinoids and other plant-derived materials, such as terpenes, flavonoids, etc. or cannabinoids may be purified substances, and may be amorphous or exist in one or more different crystalline states (polymorphs). See U.S. Pat. Nos. 7,169,942; 10,221,164; 7,169,942; 10,221,164; 7,759,526; 4,228,169; 7,179,800; and US Pat. Appln. Nos. 2006/0183922; 2005/000990; 2004/0087590. Carboxylated phytocannabinoids (carboxylated cannabinoids) refer to cannabigerolic acid (CBGA), cannabichromenic acid (CBCA), (−)-trans-Δ9-tetrahydrocannabinoic acid (Δ9-THCA or THCA), Δ8-tetrahydrocannabinoic acid (Δ8-THCA), cannabidivarinic acid, and to other phytochemical carboxylated precursors to cannabinoids, or to carboxylic acid esters of said carboxylated cannabinoids such as cannabidiolic acid methyl ester (CBDA-ME) HU-580. In some embodiments, the composition comprising CBDA may contain one or more of acidic cannabinoids and/or cannabinoids. In some embodiments, the composition comprising CBDA may contain one or more of Δ8-THCA, HU-580, CBGA, CBD, cannabidivarinic acid, and/or CBG. In preferred embodiments, CBD is hemp-derived and/or contain less 0.3% THC by weight.

The concentration of a cannabinoid or an acidic cannabinoid, or a mixture of two or more cannabinoids or acidic cannabinoids in a composition comprising CBDA may be approximately 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, or 40 mg/mL. Alternatively, the concentration of a cannabinoid, or a mixture of two or more cannabinoids or acidic cannabinoids, in a formulation may be 1 mg/g, 2 mg/g, 3 mg/g, 4 mg/g, 5 mg/g, 6 mg/g, 7 mg/g, 8 mg/g, 9 mg/g, 10 mg/g, 11 mg/g, 12 mg/g, 13 mg/g, 14 mg/g, 15 mg/g, 16 mg/g, 17 mg/g, 18 mg/g, 19 mg/g, 20 mg/g, 21 mg/g, 22 mg/g, 23 mg/g, 24 mg/g, 25 mg/g, 26 mg/g, 27 mg/g, 28 mg/g, 29 mg/g, 30 mg/g, 31 mg/g, 32 mg/g, 33 mg/g, 34 mg/g, 35 mg/g, 36 mg/g, 37 mg/g, 38 mg/g, 39 mg/g, or 40 mg/g.

In yet further embodiments, a phosphodiesterase type 5 (PDE-5) inhibitor such as, for example, sildenafil, tadalafil, vardenafil, avanafil, udenafil, mirodenafil, or lodenafil may be added to the CBDA-containing composition.

In yet other embodiments, the subject is concurrently undergoing a treatment with a PDE-5 inhibitor (e.g., sildenafil, tadalafil, vardenafil, udenafil, mirodenafil, or lodenafil). Such a PDE-5 inhibitor may be administrated orally or topically as a separate topical dosage form before, concurrently, or after the application of the CBDA-containing composition, or such a PDE-5 inhibitor and CBDA may be formulated in the same dosage form.

In another embodiment, another direct smooth muscle relaxant such as, for example, prostaglandin E1, papaverine, or minoxidil, can be added to the CBDA-containing composition.

In another embodiment, an alpha-blocker (e.g., phentolamine) can be added to the CBD-containing composition.

In another embodiment, flibanserin can be added to the CBD-containing composition to augment sexual desire.

In another embodiment, bremelanotide can be added to the CBD-containing composition to augment sexual desire.

In another embodiment, compositions comprising CBDA may include proanthocyanidins (e.g. Pycnogenol), derived from the Mediterranean or Southeast Asian pine trees (e.g. Pinus massoniana) or other sources; icariin; arginine or its salts such as the HCl salt.

As used herein, the term “phospholipid” or “phospholipids” refers to amphiphilic compounds comprising at least one saturated or unsaturated hydrophobic fatty acid moiety and a hydrophilic moiety comprising a phosphate group. These include, for example, dicetyl phosphate, soya phosphatidylcholine (SPC), egg phosphatidylcholine (EPC), hydrogenated soya phosphatidylcholine (HSPC), soya lecithin, hydrogenated soya lecithin, sphingomyelin, dioleoyl phosphatidylcholine (DOPC), dilinoleoyl phosphatidylcholine (DLPC), dioleoyl phosphatidylethanolamine (DOPE), dimyristoyl phosphatidylethanolamine (DMPE), dipalmitoyl phosphatidylethanolamine (DPPE), dimyristoyl phosphatidylcholine (DMPC), dipalmitoyl phosphatidylcholine (DPPC), distearoyl phosphatidylcholine (DSPC), dilauroyl phosphatidylcholine (DLPC), 1-myristoyl-2-palmitoyl phosphatidylcholine, 1-palmitoyl-2-myristoyl phosphatidylcholine, 1-palmitoyl phosphatidylcholine, 1-stearoyl-2-palmitoyl phosphatidylcholine, dipalmitoyl sphingomyelin, distearoyl sphingomyelin, soya phosphatidylinositol (SPI), hydrogenated phosphatidylinositol (HPI), dimyristoyl phosphatidylglycerol (DMPG), dipalmitoyl phosphatidylglycerol (DPPG), distearoyl phosphatidylglycerol (DSPG), dimyristoyl phosphatidic acid (DMPA), dipalmitoyl phosphatidic acid (DPPA), dimyristoyl phosphatidylserine (DMPS), dipalmitoyl phosphatidylserine (DPPS), hydrogenated soya phosphatidylglycerol, dioleoyl phosphatidylglycerol (DOPG), distearoyl phosphatidic acid (DSPA), and mixtures thereof, and salts thereof, preferably sodium or ammonium salts. Phospholipids may be present, on weight-to-weight (w/w) basis relative to total weight of a composition, at a level of 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or 25%. In preferred embodiments, the phospholipid is one of or is a combination of two or more of SPC, EPC, HSPC, or DSPC. In general, it may be desirable that phospholipids are condom-compatible. In certain embodiments, the liposome constituent lipids do not include cholesterol or its derivatives. In some embodiments, the lipids consist of, or consist essentially of, of the phospholipids recited above, or a subset thereof.

As used herein, the term “cryoprotectant” or “cryoprotectants” or “bulking agent” or “bulking agents” refers to compounds such as, for example, mannitol, sorbitol, lactose, trehalose, sucrose, dextran of different molecular weights such as dextran 40, inulin, glycine, L-arginine, α-cyclodextrin, β-cyclodextrin, γ-cyclodextrin, hydroxypropyl-β-cyclodextrin, hydroxypropyl-γ-cyclodextrin, randomly methylated-β-cyclodextrin, sulfobutyl ether β-cyclodextrin (SBE R-CD), hydroxypropyl methylcellulose (HPMC, hypromellose), methylcellulose, polyvinylpyrrolidone (PVP) K15, K16-18, K30, or K90, citric acid, sodium citrate, poloxamer 188 (Pluronic® F-68), poloxamer 407 (Pluronic® F-127), or polyvinyl caprolactam-polyvinyl acetate-polyethylene glycol graft copolymer (Soluplus®).

As used herein, the term “stabilizer” or “stabilizers” refers to, for example, ascorbic acid, ascorbate salts such as sodium or potassium ascorbate, citric acid, citrate salts such as, for example, sodium or potassium citrate, ethylenediaminetetraacetic acid (EDTA), EDTA salts such disodium EDTA, dipotassium EDTA, trisodium EDTA, tetrasodium EDTA, or calcium disodium EDTA, hydroxyethyl ethylenediamine triacetic acid (HEDTA), trisodium HEDTA, diethylenetriaminepentaacetic acid (DTPA), ethylenediamine-N,N′-disuccinic acid (EDDS), trisodium EDDS, DTPA pentasodium salt (pentasodium diethylenetriaminepentaacetate), methylglycinediacetic acid, trisodium dicarboxymethyl alaninate, d-glucono-1,5-lactone, gluconic acid and its salts such as sodium or potassium gluconate, or calcium gluconate, iminodisuccinic acid tetrasodium salt (tetrasodium iminodisuccinate), α-tocopherol, α-tocopherol acetate, ascorbyl palmitate, ascorbyl stearate, butylated hydroxytoluene (BHT), or butylated hydroxyanisole (BHA).

As used herein, the term “water-miscible solvent”, “water-miscible solvents”, “water-soluble solvent”, or “water-soluble solvents” refers to compounds such as, for example, ethyl alcohol (ethanol), t-butyl alcohol (t-butanol, tert-butanol, or TBA), polyethylene glycols (PEGs or macrogols) of different molecular weights such as PEG 300, PEG 400, PEG 600, PEG 1500, glycerin, diethylene glycol monoethyl ether (Transcutol®, diethylene glycol ethyl ether or 2-(2-ethoxyethoxy)ethanol), triacetin (glycerin triacetate), and propylene glycol (PG), which solvents may be used alone or as a combination of two or more solvents, with water-miscible solvents comprising, on weight-to-weight (w/w) basis relative to total weight of a formulation of 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%. In general, the compositions of the invention contain no more than 20% of PG, no more than 20% of glycerin, and no more than 20% of both PG and glycerin when both are present. Preferably, the compositions of the invention contain 6-20%, 8-18%, 6-16%, 6-14%, 8-16%, 8-14%, or 8-12% of PG. Likewise, the compositions of the invention contain 6-20%, 8-18%, 6-16%, 6-14%, 8-16%, %, or 8-12% of glycerin.

As user herein, the term “antimicrobial agent”, or “antimicrobial agents”, or “antimicrobial”, or “antimicrobials”, or “preservative”, or “preservatives” refers to substances that inhibit growth or kill microorganisms, whether antibacterial and/or antifungal agents, such as, for example, methyl paraben (methylparaben), ethyl paraben (ethylparaben), propyl paraben (propylparaben), butyl paraben (butylparaben), and heptyl paraben (heptylparaben), benzoic acid and benzoic acid salts such as sodium benzoate, dehydroacetic acid and sodium dehydroacetate, sorbic acid and its salts such as sodium sorbate, salicylic acid and its salts such as sodium salicylate, p-anisic acid, caprylhydroxamic acid, caprylic acid and its salts such as sodium caprate, levulinic acid and its salts such as sodium levulinate, undecylenic (10-undecenoic) acid and its salts such as sodium undecylenate, eugenol, menthol, 1,2-pentanediol, 1,2-hexanediol, 1,2-octanediol, 1,2-decanediol, ethylhexylglycerin, glyceryl caprate, glyceryl caprylate, glyceryl undecylenate, phenethyl alcohol, and phenylpropanol. The antimicrobial agents, whether used singly or as a blend of two or more antimicrobial agents, are to be used in the concentrations that vary from agent to agent and are to be introduced into the formulations in either organic or aqueous phase, all of which is known to those skilled in the art. As used herein, the term “thickener” or “thickening agent” refers to substances, whether gelling or non-gelling, which raise viscosity and which may or may not require pH adjustment or addition of salts (ions) to produce increase in viscosity. Examples of “thickeners” or “thickening agents” are crosslinked polyacrylic acid polymers such as Carbopol® 71 G, 940, 971 P, 974P, 980, 981, 5984 EP, ETD 2020, Ultrez 10, Pemulen™ TR-1 and TR-2 NF polymers; hydroxyethyl acrylate/sodium acryloyldimethyl taurate copolymers; polyacrylate crosspolymer-6; sodium acrylate/acryloyldimethyltaurate/dimethylacrylamide crosspolymer; hyaluronic acid of average molecular weights of approximately 8,000-13,000, 50,000-75,000, 450,000-500,000, or one million or more Da; hydroxypropyl methylcellulose (HMPC, hypromellose, substitution types 2910, 2208, or 2906) in grades of viscosity of 2% aqueous solution of approximately 3 cP, 4 cP, 5 cP, 15 cP, 50 cP (40-60 cP), 100 cP (80-120 cP), 200-300 cP, 500-1000 cP, 1000-2000 cP, 4000 cP, methylcellulose, hydroxyethyl cellulose in grades of viscosity of 5% aqueous solution of 100 cP, 50-150 cP, of 2% aqueous solution at 20° C. of 200-300 cP, 800-1500 cP, approximately 2000 cP, approximately 3400 cP, or 5000 cP, ethylcellulose, hydroxypropyl cellulose, also gums such as xanthan gum, locust bean gum, guar gum, algin (alginic acid), as well as agar gum, pectin, K-carrageenan, i-carrageenan, as well as starches such as potato starch, corn (maize) starch, wheat starch, or pea starch. Some of the thickeners are multifunctional substances and in certain compositions a thickener may act as an anti-caking agent and/or a lubricating agent, and/or a humectant. As used herein, the term “lubricating agent” may refer to a thickener or it may refer to a substance that is not a thickener, for example, to lauric acid and its salts such as sodium laurate, or isopropyl myristate.

As used herein, the term “formulation” is used interchangeably with the term “composition.” In general, a “formulation” of the invention comprises CBDA and, optionally, one or more cannabinoids or carboxylated cannabinoids, and may contain one or more of phospholipids, surfactants, cryoprotectants, bulking agents, stabilizers, water-miscible solvents, oils, water-immiscible solvents, hard fats, butters, anti-microbial agents, and thickeners. The compositions described herein are intended for use in pharmaceutical, phytopharmaceutical, nutraceutical, cosmetic, or veterinary settings by various routes of administration, such as dermal (topical or transdermal), mucosal (vaginal or rectal), or and may be formulated as an ointment, a cream, a suspension, a lotion, a paste, a gel, a balm, or a suppository, or in soft- or hard-shell capsules, or tinctures, or fluids of different viscosities, or serums, the basic preparation techniques of which are known to those skilled in the art.

As used herein, the term “application” or “applying”, or “administration”, or “administering” means placing or spreading or rubbing on a quantity of a composition to female subject's genital area(s), such as on or around external genitalia, for example, onto absorptive mucosa, comprising one or more of: the introitus, the vulva, the labia minora, the clitoris and the vaginal vault, or placing a suppository inside a vagina using an applicator or without an applicator, or using a vaginal lubricant by placing or spreading a quantity of a composition on or around external genitalia, inside the vagina, or onto an object to be used for penetrative sexual activity. The term “sexual activity” refers to sexual intercourse or other stimulation with a partner or masturbation.

The following examples are not intended to be limiting. Those of skill in the art will, in light of the present disclosure, appreciate that many changes can be made in the specific materials and which are disclosed and still obtain a like or similar result without departing from the spirit and scope of the invention.

### EXAMPLES

### Example 1. In Vitro Isometric Tension Experiments on Vaginaltissue

The following in vitro methodology was utilized to examine the concentration-dependent response of rat vaginal tissue, both proximal and distal, to CBDA and CBD following contraction by electrical field stimulation (similar to efferent neurostimulation in vivo).

### Tissue Preparation

Intact female Sprague-Dawley rats were anesthetized and vagina harvested. A maximum of eight vaginal strips (4 proximal and 4 distal) were obtained from each rat in circular direction and mounted in organ chambers filled with an oxygenated physiological salt solution.

### Example 2. Evaluation of the Smooth Muscle Reactivity with Isolated Organ Baths

Strips were excised from the tissue samples and connected to force transducers for isometric tension recording. Organ baths were filled with Krebs buffer maintained at 37° C. and bubbled with 95% O₂ and 5% CO₂, pH 7.4.

After the equilibration period, the strips were primed first by the addition to the organ bath of KCl (100 mM, 10 min) and then after a washing step, by stimulation to EFS (electrical parameters: 20 Hz, 3 ms, 10 s, 300 mA). After this priming period, the strips were washed and a baseline frequency response curve (FRC) was obtained (3 ms, 10 s, 300 mA, frequency at 1, 2, 4, 8, 16, 32, and 48 Hz, every 2 minutes). Then, 6 strips (3 proximal and 3 distal) were incubated with vehicle for 10 minutes before applying a second FRC. The remaining 2 strips (one proximal and one distal) served as a time-controls. After a washing step, a third FRC was applied after the incubation of the test compound at 1 μg/mL for a period of 10 min. This was repeated with the concentration of 10 μg/mL, and 100 μg/mL.

### Example 3. A Dose-Response (Concentration Response) Relaxation of Rat Vaginal Smooth Muscle Tissue by Cannabidiol (CBD)

The experiments were performed following the general procedures described in Examples 1 and 2. The data are mean±SEM of N experiments using vaginal strips from N different rats. p=ns, * p<0.05, ** p<0.01, *** p<0.001, **** p<0.0001, two-way ANOVA.

Compared to the vehicle control, CBD induced a statistically-significant relaxation of EFS-induced contractions of proximal vagina strips at the concentrations of 10 and 100 μg/mL (FIG. 1A). Compared to the vehicle control, CBD induced a statistically-significant relaxation of EFS-induced contractions of distal vagina strips at the concentrations of 1, 10, and 100 μg/mL (FIG. 1B).

### Example 4. a Dose-Response (Concentration Response) Relaxation of Rat Vaqinal Smooth Muscle Tissue by Cannabidiolic Acid (CBDA)

The experiments were performed following the general procedures described in Examples 1 and 2. The data are mean±SEM of N experiments using vaginal strips from N different rats; p=ns, * p<0.05, ** p<0.01, *** p<0.001, **** p<0.0001, two-way ANOVA.

Compared to the vehicle control, CBD induced a statistically significant relaxation of EFS-induced contractions of distal vagina strips at the concentrations of 1, 10, and 100 μg/mL (FIG. 2A). Compared to the vehicle control, CBDA induced a statistically-significant relaxation of EFS-induced contractions of distal vagina strips at the concentrations of 10 and 100 μg/mL (FIG. 2B).

### Example 5. Comparison of the Concentration-Dependent Pharmacological Relaxant Effects of CBD and CBDA on Rat Vaginal Smooth Muscle Tissue

The comparison plots of the pharmacological relaxant effects of CBD and CBDA on rat vaginal smooth muscle tissue are shown in FIGS. 3, 4, and 5. Also, shown are the results of the evaluation of the differences in effects exerted by CBD and CBDA for statistical significance. The data are mean±SEM of N experiments using vaginal strips from N different rats; p=ns, * p<0.05, ** p<0.01, *** p<0.001, **** p<0.0001, two-way ANOVA.

The pharmacological relaxant effect induced by CBDA was stronger than that induced by CBD, which difference was clearly statistically significant for both proximal and distal tissues at concentrations of 10 μg/mL and 100 μg/mL (FIGS. 4A, 4B, 5A, and 5B). CBDA also was stronger at relaxing the proximal vaginal smooth muscle at 1 μg/mL (FIG. 3A) whereas there was no statistically significant difference between CBDA and CBD at 1 μg/mL for the relaxation of distal vaginal smooth muscle (FIG. 3B).

1. A method of improving female sexual function and/or treating a female sexual disorder, the method comprising applying a topical composition to a surface of a female subject's genital area(s), said topical composition comprising cannabidiolic acid (CBDA) and, optionally, one or more additional carboxylated or decarboxylated cannabinoids, the topical composition being applied to mucosal surface prior to sexual activity in the amount and for a period of time sufficient for a sexual function of the subject to be enhanced and/or the disorder to be ameliorated during the sexual activity.

2-26. (canceled)

27. A method of effecting relaxation of female genital smooth muscles in a subject in need thereof, the method comprising:
applying a topical composition comprising cannabidiolic acid (CBDA) to a surface of a female subject's genital area(s) prior to sexual activity in an amount and for a time sufficient to relax female genital smooth muscles, thereby enhancing a female sexual function and/or ameliorating a female sexual disorder.

28. The method of claim 27, wherein the surface of the subject's genital area comprises absorptive mucosa, and wherein the genital area comprises one or more of the introitus, the vulva, the labia minora, the clitoris, and the vaginal vault.

29. The method of claim 27, wherein the topical composition is applied intravaginally.

30. The method of claim 27, wherein enhancement of the sexual function and/or amelioration of the disorder is measured by vaginal and clitoral smooth muscle relaxation and/or increased vaginal and clitoral blood flow.

31. The method of claim 27, wherein the subject is premenopausal or post-menopausal.

32. The method of claim 27, wherein a total amount of CBDA in the applied topical composition is from 10 mg to 100 mg of CBDA.

33. The method of claim 27, wherein the topical composition is applied 5 min to 40 min prior to the sexual activity.

34. The method of claim 27, wherein the topical composition further comprises CBD, CBG, cannabidivarinic acid, or CBGA.

35. The method of claim 27, wherein the topical composition is in a form of a vaginal suppository.

36. The method of claim 27, wherein the subject suffers from a disorder selected from the group consisting of Sexual Interest/Arousal Disorder (SIAD) and Female Orgasmic Disorder.

37. The method of claim 27, wherein the topical composition further comprises a phosphodiesterase type 5 inhibitor.

38. The method of claim 27, wherein the inhibitor is selected from the group consisting of sildenafil, tadalafil, vardenafil, avanafil, udenafil, mirodenafil, and lodenafil.

39. The method of claim 27, wherein topical composition further comprises an additional smooth muscle relaxant.

40. The method of claim 39, wherein the additional muscle relaxant is selected from the groups consisting of prostaglandin E1, papaverine, and minoxidil.

41. The method of claim 27, wherein topical composition further comprises an alpha-blocker.

42. The method of claim 41, wherein the alpha-blocker is phentolamine.
